(19) United States
(12) Patent Application Publication
(10) Pub. No.: US 2018/0000361 A1
(43) Pub. Date: Jan. 4, 2018

(54) BLOOD PRESSURE MONITOR

(71) Applicant: Empire Technology Development LLC, Wilmington, DE (US)

(72) Inventor: Zhen Xiao, Beijing (CN)

(73) Assignee: Empire Technology Development LLC, Wilmington, DE (US)

(21) Appl. No.: 15/540,317

(22) PCT Filed: Jan. 4, 2015
(86) PCT No.: PCT/CN2015/070026

§ 371 Date: Jun. 28, 2017

(51) Int. Cl. A61B 5/022; A61B 5/00

(52) U.S. Cl. CPC A61B 5/02241; A61B 5/681; A61B 5/0022; A61B 5/0059; A61B 5/6803

(57) ABSTRACT

Technologies are generally described for systems, devices and methods relating to blood pressure monitors. Blood pressure monitors may include a processor and a structure effective to be in communication with the processor. The structure may be effective to apply pressure to an object. Blood pressure monitors may include at least one photoreceptor effective to be in communication with the processor. Photoreceptors may be located in the device so as to be effective to detect light returned from the object while the pressure is applied. Blood pressure monitors may be effective to determine color values of the returned light, determine changes in the color values of the returned light, and determine a blood pressure based on the changes in the color values.

### BACKGROUND

Unless otherwise indicated herein, the materials described in this section are not prior art to the claims in this application and are not admitted to be prior art by inclusion in this section.

Blood pressure may be the pressure exerted by blood in the circulatory system on the walls of blood vessels. Blood pressure may be measured in terms of systolic pressure and diastolic pressure. Systolic pressure may be peak pressure in the arteries, and may occur when the ventricles of the heart are contracting. Diastolic pressure may be minimum pressure in the arteries, and may occur when the ventricles of the heart are full of blood.

### SUMMARY

In some examples, devices are generally described. In various examples, the devices may include a processor. In some other examples, the devices may include a structure effective to be in communication with the processor. In some examples, the structure may be effective to apply pressure to an object. In some other examples, the devices may include at least one photoreceptor effective to be in communication with the processor. In various examples, the at least one photoreceptor may be located in the device and may be effective to detect light returned from the object while the pressure is applied. In some examples, the processor may be further effective to determine color values of the returned light. In other examples, the processor may be further effective to determine changes in the color values of the returned light. In various other examples, the processor may be further effective to determine a blood pressure based on the changes in the color values.

In some examples, methods to determine a blood pressure are generally described. In various examples, the methods may include applying, with a structure, a pressure to an object. In some other examples, the methods may further include detecting, by at least one photoreceptor, light returned from the object. In some examples, the returned light may be detected while the pressure is applied to the object. In some other examples, the methods may further include determining, by a processor, color values of the returned light that has been detected. In various other examples, the methods may further include determining, by the processor, changes in the color values of the returned light that has been detected. In still other examples, the methods may further include determining, by the processor, the blood pressure based on the changes in the color values.

In some other examples, methods to determine blood pressure are generally described. In various examples, the methods may include depressing skin of a subject with a structure at a first pressure. In some other examples the methods may further include detecting, by a photoreceptor, first returned light from the subject. In some examples, the first returned light may be detected while the first pressure is applied to the skin. In various other examples, the methods may further include determining, by a processor, a first color of the first returned light. In yet other examples, the methods may further include determining, by the processor, a first color change based on a first change in the first color of the first returned light. In some examples, the methods may include depressing the skin of the subject with the structure at a second pressure different from the first pressure. In various other examples, the methods may further include detecting, by the photoreceptor, second light returned from the subject, wherein the second returned light is detected while the second pressure is applied to the skin. In some other examples, the methods may further include determining, by the processor, a second color of the second returned light. In various examples, the methods may further include determining, by the processor, a second color change based on a second change in the second color of the second returned light. In some examples, the second color change may be different from the first color change. In some examples, the methods may further include determining the blood pressure based on the first color change and the second color change.

In some other examples, apparatuses to monitor blood pressure are generally described. In some examples, the apparatuses may include a surface. In some other examples, the apparatuses may include a structure configured to deploy from the surface and effective to apply pressure to a skin of a subject in response to the surface being in proximity to the skin. In some further examples, the apparatuses may include a light source effective to emit first light toward the subject. In various other examples, the apparatuses may further include at least one photoreceptor. In some examples, the at least one photoreceptor may be coupled to the surface and located on the surface so as to be effective to detect second light returned from the subject while the pressure is applied. In some examples, the at least one photoreceptor may be effective to generate an output electrical signal that may represent the detected second light. In some other examples, a color of the second light may change in response to the applied pressure. In various other examples, the at least one photoreceptor may be further effective to change a characteristic of the output electrical signal in response to the changed color of the second light. In various examples, the changed characteristic of the output electrical signal may be useable to determine a blood pressure of the subject.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 illustrates an isometric view of an example device that can be used to implement a blood pressure monitor;

FIG. 2 depicts a side view of the example device of FIG. 1 in contact with skin of a subject;

FIG. 3 depicts a side view of the example device of FIGS. 1 and 2 with additional details related to the measurement of blood pressure;

FIG. 4 depicts an example coloration pattern which may be used to measure blood pressure;

FIG. 5 depicts an isometric view of an example device that can be used to implement a blood pressure monitor;

FIG. 6 depicts a flow diagram for an example process to operate a blood pressure monitor;

FIG. 7 illustrates an example computer program product that can be utilized to operate a blood pressure monitor;

FIG. 8 is a block diagram illustrating an example computing device that is arranged to implement or otherwise operate in conjunction with a blood pressure monitor;

all arranged according to at least some embodiments described herein.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. The aspects of the present disclosure, as generally described herein, and illustrated in the drawings, can be arranged, substituted, combined, separated, and designed in a wide variety of different configurations.

Briefly stated, technologies are generally described for systems, devices and methods relating to blood pressure monitors. Blood pressure monitors may include a processor and a structure effective to be in communication with the processor. The structure may be effective to apply pressure to an object. Blood pressure monitors may include at least one photoreceptor effective to be in communication with the processor. Photoreceptors may be located in the device so as to be effective to detect light returned from the object while the pressure is applied. Blood pressure monitors may be effective to determine color values of the returned light, determine changes in the color values of the returned light, and determine a blood pressure based on the changes in the color values.

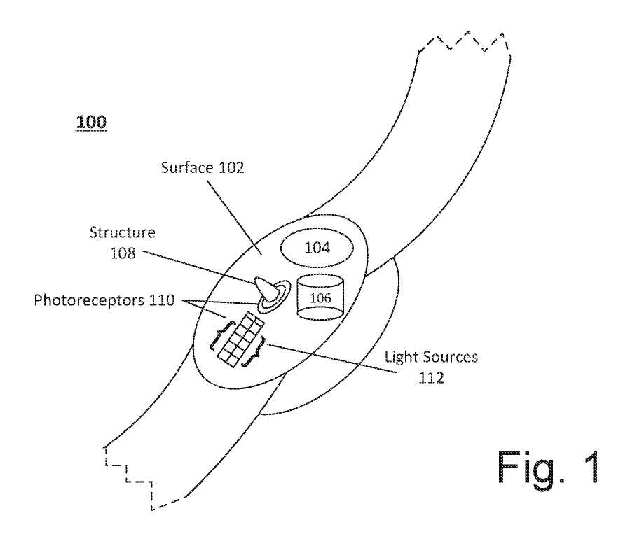

FIG. 1 illustrates an example device 100 that can be used to implement a blood pressure monitor arranged according to at least some embodiments described herein. In some examples, device 100 may be a form of wearable technology such as a smart watch, or augmented reality device, such as augmented reality glasses. In some other examples, device 100 may be implemented as a medical device such as a bracelet, wristband, patch, sleeve, or other device which may be placed in contact with or otherwise in close proximity to skin of a subject or object to measure or otherwise determine blood pressure.

Device 100 may include computing elements such as a processor 104 and a memory 106 effective to be in communication with processor 104. Device 100 may further include a surface 102, one or more photoreceptors 110, and/or one or more light sources 112. Device 100 may include a structure 108. Structure 108 may be effective to apply pressure to an object when device 100 is placed in contact with or otherwise in close proximity to the object. In some examples, structure 108 may be implemented with a needle with a dull point and may be retracted into surface 102 such that surface 102 may be a flat, planar surface when structure 108 is in a retracted position. In some examples, surface 102, or portions of surface 102, may be transparent or translucent. Structure 108 may be formed in the shape of a cone, an ellipsoid, a cylinder, a cylinder with a hemispherical top or bottom, or other shape. In various examples, structure 108 may be designed in such a way as to apply pressure to skin of a user of device 100 without piercing the skin of the user. In some embodiments, processor 104 and/or memory 106 may be located externally to device 100, such that the other depicted components of device 100 can communicate in a wired or wireless manner with the external processor 104 and/or memory 106.

In some examples, photoreceptors 110 and/or light sources 112 may be arranged so as to linearly extend away from structure 108 along surface 102. In some other examples, photoreceptors 110 and/or light sources 112 may be arranged annularly around structure 108. In some examples, light sources 112 may include light emitting diodes and/or lasers. In various examples, photoreceptors 110 may include charge coupled devices (“CCDs”) or complementary metal oxide semiconductor (“CMOS”) devices. In some further examples, photoreceptors 110 and/or light sources 112 may be arranged within device 100, such that photoreceptors 110 and/or light sources 112 surround or partially surround structure 108. Other arrangements are possible. Light sources 112 may be effective to emit one or more wavelengths of light, including light in the visible wavelength range, ultraviolet range, infrared wavelength range or other wavelength range, such as light provided by a laser designed to emit light of a particular wavelength. Photoreceptors 110 may be effective to detect light returned from an object when surface 102 is placed against the object. In various examples, photoreceptors 110 may be coupled to and/or located on surface 102 of device 100. Photoreceptors 110 may be effective to detect light returned from an object and generate an output electrical signal which may represent the detected light. The output electrical signal may represent various characteristics of the detected light. In some examples, characteristics of the detected light may include wavelength, frequency, and magnitude. In some examples, photoreceptors 110 may be effective to change a characteristic of an output electrical signal in response to changes in color or intensity of the detected light. In some examples, processor 104 may control operation and/or activation of light sources 112 and/or photoreceptors 110. For example, processor 104 may activate light sources 112 and/or photoreceptors 110 in response to detection that structure 108 has been pressed against the skin of a subject.

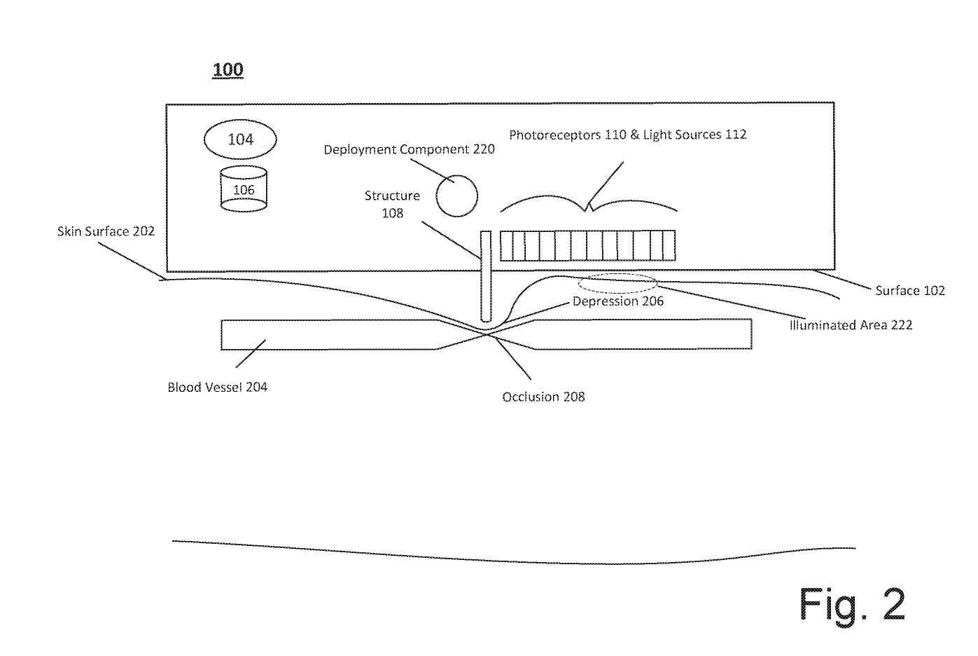

FIG. 2 depicts a side view of device 100 of FIG. 1 in contact with skin of a subject arranged in accordance with various embodiments described herein. Those components in FIG. 2 that are labeled identically to components of FIG. 1 will not be described again for the purposes of clarity and brevity.

A user of device 100 may measure or otherwise determine the blood pressure of a subject (who may also be a user) through activation of a blood pressure monitor feature of device 100. A skin surface 202 of a subject may be placed against or otherwise in close proximity to surface 102 of device 100 such that skin surface 202 abuts or is otherwise near surface 102. A user of device 100 (the user may or may not be the subject) may activate the blood pressure monitor feature of device 100 through, for example, a user interface. In other examples, device 100 may be effective to periodically or otherwise repeatedly measure blood pressure of a subject. For example, device 100 may be implemented in a smart watch. Device 100 may measure blood pressure of the subject wearing the smart watch every X hours or minutes, for instance. In some examples, processor 104 may execute one or more instructions stored in memory 106 to activate a blood pressure monitor feature of device 100. In response to activation of the blood pressure monitor feature, a deployment component 220 coupled to structure 108 may extend structure 108 away from surface 102 and toward skin surface 202 of the subject. Alternatively or additionally to deployment component 220, other components may be coupled to structure 108 and configured to deploy (e.g., extend and/or retract) structure 108, such as a motor, springs, hydraulic mechanisms, gears, air pressure devices, electromagnetic devices, etc. Structure 108 may apply pressure to skin surface 202 at a constant or otherwise suitable rate, while extending and extended, to form a depression 206 in skin surface 202. As a result of depression 206, one or more blood vessels 204 under the skin of the subject may become occluded. In the example shown in FIG. 2, occlusion 208 of blood vessels 204 may result from structure 108 forming depression 206 in skin surface 202. In some examples, blood vessels 204 may be arteries, veins, venules, arterioles or capillaries. When blood vessel 204 becomes occluded, blood flow may be at least partially blocked in the area surrounding occlusion 208. While blood vessel 204 is occluded, light sources 112 may emit light toward skin surface 202 to produce an illuminated area 222 of skin surface 202. In some examples, light sources 112 may produce illuminated area 222 in areas surrounding or nearby depression 206. Photoreceptors 110 may detect light that is returned from skin surface 202, blood vessel 204, and blood inside blood vessel 204 in response to the light emitted from light sources 112. In some examples, light returned from skin surface 202, blood vessel 204, and blood inside blood vessel 204 may be reflected light and may have a different wavelength and/or intensity relative to light emitted by light sources 112. Photoreceptors 110 may be positioned at various distances and orientation/location from structure 108 so that light returned from different positions on skin surface 202 may be measured or otherwise analyzed. As will be discussed in further detail below, device 100 may determine systolic and diastolic blood pressures based upon the color of light detected by photoreceptors 110 while structure 108 forms depression 206.

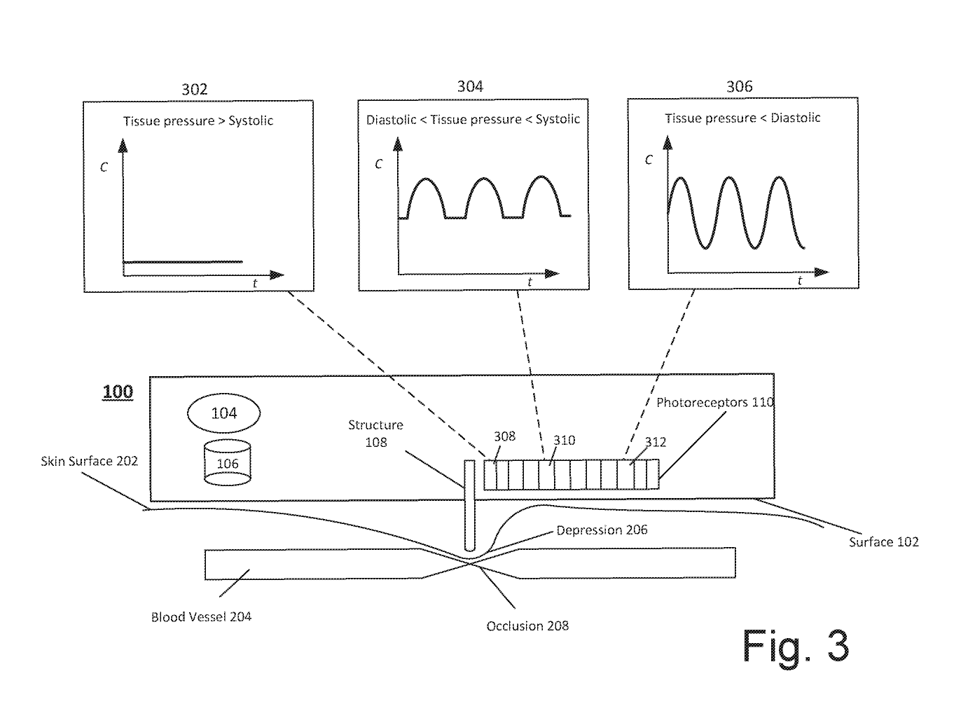

FIG. 3 depicts a side view of the example device of FIGS. 1 and 2 with additional details related to the measurement of blood pressure arranged in accordance with various embodiments described herein. Those components in FIG. 3 that are labeled identically to components of FIGS. 1 and 2 will not be described again for purposes of clarity and brevity.

As is described in further detail below, when structure 108 forms depression 206, color values C may be determined by processor 104, based on electrical signals output by photoreceptors 110 based on detected light. Processor 104 may determine color values C for electrical signals output by photoreceptors 110 located at various distances from central point of depression 206. Color values C may be measured or otherwise determined along a red-blue color axis, where the red direction, indicating oxygenated blood, may be the positive direction and the blue direction, indicating deoxygenated blood, may be the negative direction. Processor 104 may calculate the change of the color values C with respect to time.

For purposes of the discussion below, a tissue pressure may be the resultant pressure exerted upon skin surface 202 at various positions as a result of structure 108 applying pressure to form depression 206. Processor 104 may calculate the tissue pressure along various portions of skin surface 202 based on the application of pressure from structure 108. Processor 104 may identify various characteristic functions which may relate color values of light detected by photoreceptors 110 to time. As depicted by an example characteristic function 302, at a first photoreceptor position 308, color values C may be constant with respect to time and may have smaller values relative to the values of C at other photoreceptor positions 310, 312. Processor 104 may determine that the tissue pressure at a portion of skin surface 202 that corresponds to photoreceptor position 308 may be greater than systolic pressure. As depicted by an example characteristic function 306, at a second photoreceptor position 312, color values C may vary with a cycle of the heart rate of the subject. As a result, processor 104 may determine that a tissue pressure at the portion of skin surface 202 which corresponds to photoreceptor position 312 may be less than diastolic pressure. As depicted by an example characteristic function 304, at a third photoreceptor position 310, color values C may vary periodically with a constant or semi-constant period with respect to time. As a result, processor 104 may determine that tissue pressure at a portion of skin surface 202 which corresponds to photoreceptor 310 may be less than systolic pressure and greater than diastolic pressure.

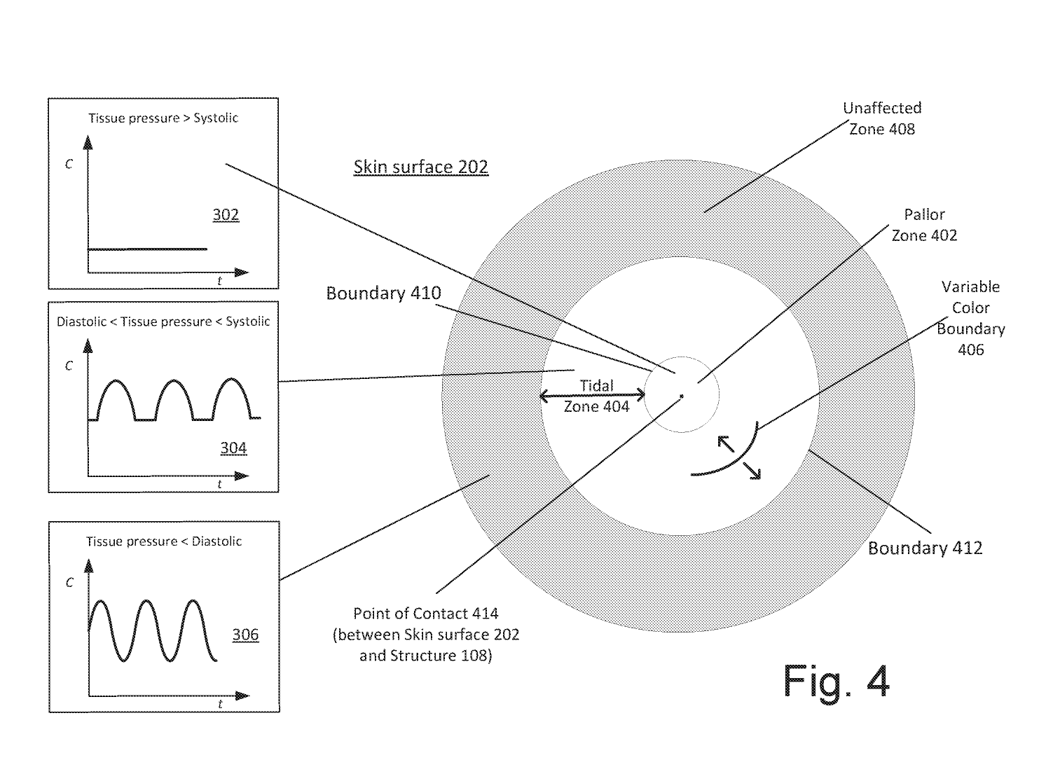

FIG. 4 depicts an example coloration pattern which may be used to measure blood pressure arranged in accordance with various embodiments described herein. Those components in FIG. 4 that are labeled identically to components of FIGS. 1-3 will not be described again for purposes of clarity and brevity.

Positions on skin surface 202 where tissue pressure is greater than systolic pressure may be a pallor zone 402. Blood may be occluded from pallor zone 402 as a result of tissue pressure exceeding systolic pressure. Therefore, C may have a low value relative to other positions which are further away from the point of contact between skin surface 202 and structure 108. Additionally, within pallor zone 402, C may be constant as no blood may enter pallor zone 402 due to the tissue pressure exceeding the systolic pressure. Therefore, C may be maintained with respect to time in the pallor zone. Processor 104 may identify the location, dimensions, and/or area of pallor zone 402 based on the positions of the particular photoreceptors 110 which exhibit or otherwise correspond to characteristic function 302.

Positions on skin surface 202 where tissue pressure is between systolic pressure and diastolic pressure may be referred to as tidal zone 404. Processor 104 may identify the location, dimensions, and/or area of tidal zone 404 based on the positions of the particular photoreceptors 110 which exhibit or otherwise correspond to characteristic function 304. Variable color boundary 406 may represent the flow of blood pulsing towards and flowing away from the point of contact 414 between skin surface 202 and structure 108. Variable color boundary 406 may move within tidal zone 404 as a result of the heart pushing blood towards pallor zone 402 and tissue pressure forcing the blood out of tidal zone 404. Blood may pulse towards point of contact 414 and may stop at a boundary 410, where tissue pressure exceeds systolic pressure. Therefore, C may vary periodically with a constant or semi-constant period in tidal zone 404. Processor 104 may identify boundary 410. Boundary 410 may be located between pallor zone 402 and tidal zone 404. Processor 104 may determine systolic blood pressure of the subject based on the tissue pressure at boundary 410.

In an unaffected zone 408, the tissue pressure may be less than diastolic pressure. Processor 104 may identify the location, dimensions, and/or start of unaffected zone 408 based on the positions of the particular photoreceptors 110 which exhibit or otherwise correspond to characteristic function 306. As tissue pressure may be less than diastolic pressure in unaffected zone 408, diastolic pressure may overcome tissue pressure as the dominant force by which blood is driven back towards the heart. The blood circulation may be unaffected and C may vary with a cycle of the heart rate of the subject in unaffected zone 408. Processor 104 may identify a boundary 412. Boundary 412 may be located between tidal zone 404 and unaffected zone 408. Processor 104 may determine diastolic blood pressure of the subject based on the tissue pressure at boundary 412, as boundary 412 may represent the point at which diastolic pressure overcomes tissue pressure generated by structure 108 as the dominant force by which blood is returned to the heart.

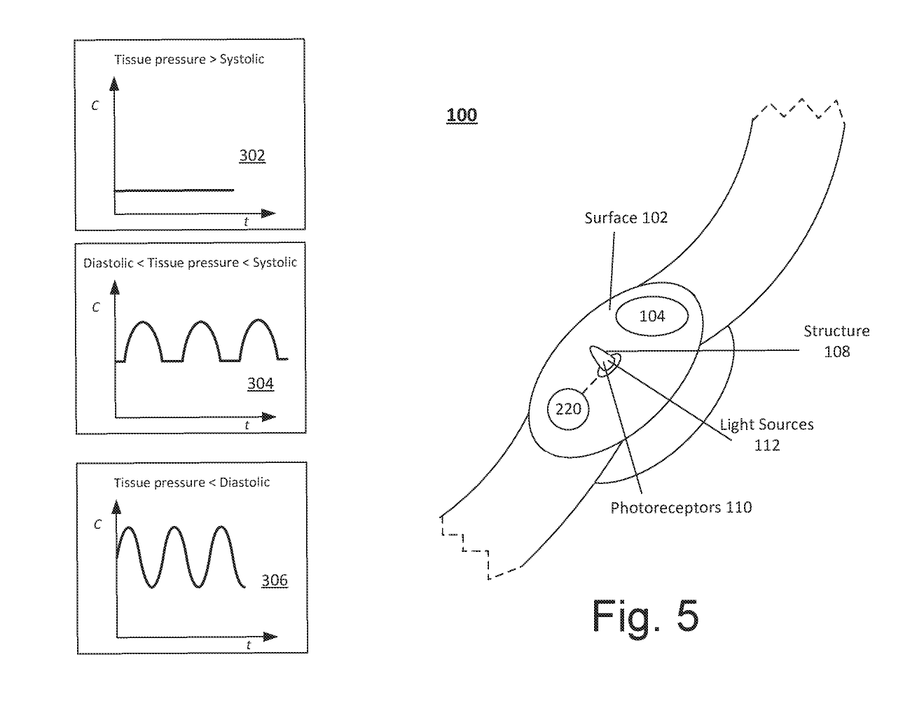

FIG. 5 depicts an isometric view of an example device 100 that can be used to implement a blood pressure monitor arranged in accordance with various embodiments described herein. Those components in FIG. 5 that are labeled identically to components of FIGS. 1-4 will not be described again for purposes of clarity and brevity.

In some examples, photoreceptors 110 and/or light sources 112 may be located within structure 108. In examples where photoreceptors 110 and/or light sources 112 are located within structure 108, portions of structure 108 may be formed from a photoconductive material, such that light emitted from light sources 112 and light detected by photoreceptors 110 may pass through the photoconductive portions of structure 108.

In various examples, deployment component 220 may be effective to extend and/or retract structure 108 such that structure 108 depresses the skin of a subject with a variable amount of pressure. For example, deployment component 220 may exert a force on structure 108 such that structure 108 applies an increasing or decreasing amount of pressure to the skin of a subject. In various examples, pressure applied to the skin of the subject may be increased or decreased in a linear or non-linear manner. As previously described above, other ways that are alternative or additional to deployment component 220 may be used to extend and/or retract structure 108.

Photoreceptors 110 may detect light returned from the subject while structure 108 applies various pressures to the skin of the subject. Processor 104 may be effective to determine color values C of the returned light at the various pressures. Processor 104 may be further effective to determine changes in the color values C with respect to time. According to various embodiments, processor 104 may be located in or on the same device shown in FIG. 5 or may be remotely positioned at a location external to the device, such that the other depicted components of the device of FIG. 5 can communicate in a wired or wireless manner with the external processor 104.

Processor 104 may determine characteristic functions 302, 304 and 306 based on the changes of the color values C with respect to time. As discussed, characteristic functions 302, 304 and 306 may correspond to various pressures applied to the skin of the subject by structure 108. Processor 104 may determine tissue pressure by dividing a particular pressure applied by structure 108 by an area of structure 108 that is pressed against a subject's skin. In some examples, the tissue pressure at which the function of C with respect to time changes from characteristic function 302 to characteristic function 304 may be equal to the systolic blood pressure of the subject. In some other examples, the tissue pressure at which the function of C with respect to time changes from characteristic function 304 to characteristic function 306 may be equal to the diastolic blood pressure of the subject.

In some examples, device 100 may be calibrated based on comparison with other blood pressure monitoring systems, such as a sphygmomanometer. In some other examples, device 100 may be worn on the subject's wrist and calibrated prior to use. The subject may place their wrist and device 100 in front of the subject's chest, level with the subject's heart. The outstretched arm of the subject may be measured. Alternatively, the arm length may be estimated based on a height of the subject. Blood pressure may be measured using the various techniques described above to measure the systolic and diastolic blood pressures of the subject while the subject's arm is raised above the subject's head. Blood pressure may be measured using the techniques described above to measure the systolic and diastolic blood pressures of the subject while the subject's arm is lowered at the subject's side. As the height of the measuring point may differ by two times the arm length the blood pressure differences may represent the hydrostatic pressure generated by two times the length of the arm. The hydrostatic pressure may be used for calibrating device 100.

Among other features, a blood pressure monitor arranged in accordance with the description herein may be effective to monitor the blood pressure of a subject at various times. The blood pressure monitor may be effective to generate an alert if the blood pressure of a subject is determined to be outside of predetermined tolerance levels. A blood pressure monitor as described herein may be inconspicuously integrated within wearable devices such as eye glasses, watches, ear-rings, rings, ear phones, finger nails, etc. The blood pressure monitor described herein may allow a user to measure the blood pressure of a subject without the use of bulky equipment such as sphygmomanometers. Additionally, the blood pressure monitors described herein may be used effectively without specialized training of the user or subject.

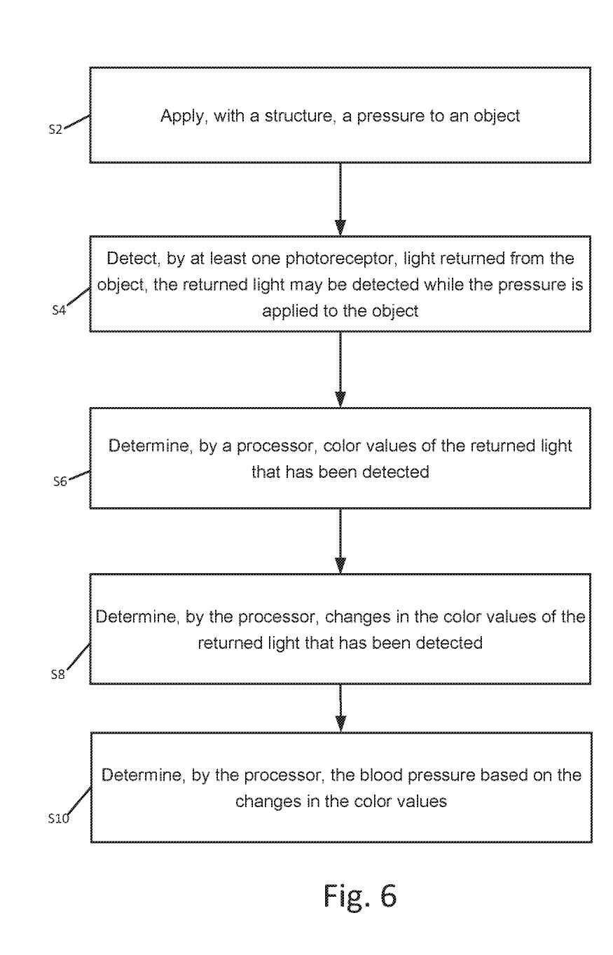

FIG. 6 depicts a flow diagram for example process to operate a blood pressure monitor, arranged in accordance with at least some embodiments described herein. In some examples, the process in FIG. 6 could be implemented using device 100 discussed above and could be used to determine a blood pressure. An example process may include one or more operations, actions, or functions as illustrated by one or more of blocks S2, S4, S6, S8 and/or S10, etc. Although illustrated as discrete blocks, various blocks may be divided into additional blocks, combined into fewer blocks, or eliminated, depending on the particular implementation. Blocks may be supplemented with additional blocks representing other operations, actions, or functions. At least some portion of the process in FIG. 6 may be used or performed by a processor, such as processor 104, as described above. In some embodiments, various other components of the devices described herein may perform some portions of the process of FIG. 6, independently of the processor or under control of the processor.

The process to operate the blood pressure monitor may begin at block S2, “Apply, with a structure, a pressure to an object.” At block S2, a structure (such as structure 108) may be used to apply pressure to an object. In some examples, the object may the skin of a subject. In various examples, the pressure may be a first pressure. In some examples, a second pressure may be applied by depressing the skin of the subject with the structure at a second pressure that may be greater than the first pressure. In some examples, the structure may be arranged in a conical shape. The structure may be effective to apply the pressure at a constant rate. In some examples, the structure may be effective to extend from, and retract into, a surface of the device to apply the pressure. In various examples, the structure may be included in augmented reality glasses such that the structure may be in contact with the object.

The process to operate the blood pressure monitor may continue from block S2 to block S4, “Detect, by at least one photoreceptor, light returned from the object, the returned light may be detected while the pressure is applied to the object.” At block S4, light returned from the object may be detected by at least one photoreceptor (such as one or more of the photoreceptors 110). The returned light may be detected while the pressure is applied to the object. For example, individual photoreceptors 110 may detect returned light from various positions of the subject's skin and produce electrical signals which may correspond to the returned light. The electrical signals may be analyzed by processor 104 to determine a color value for the returned light. In some examples, operation of the blood pressure monitor may include emitting light toward the subject from a light source (such as one or more of the light sources 112). The light source may be configured to be in communication with the processor, such that the processor can control the activation, deactivation, output light direction, output light intensity, etc. of individual light sources. In some examples, the at least one photoreceptor may be arranged inside the structure. In various other examples, the structure and/or the at least one photoreceptor may be located within a wristband. In some examples, the at least one photoreceptor may be arranged annularly around the structure. In various other examples, the at least one photoreceptor may be arranged so as to extend linearly away from the structure.

The process to operate the blood pressure monitor may continue from block S4 to block S6, “Determine, by a processor, color values of the returned light that has been detected.” At block S6, the processor may determine color values of the returned light that has been detected by the photoreceptors. For instance, the processor may determine color values by analyzing the electrical signals that are output by the photoreceptors in response to receiving the returned light. In some examples, the processor may determine color values by analyzing the frequency, wavelength and/or magnitude of the returned light. In various other examples, the processor may use a lookup table to determine color values based on factors such as the frequency and/or wavelength of the returned light.

The process to operate the blood pressure monitor may continue from block S6 to block S8, “Determine, by the processor, changes in the color values of the returned light that has been detected.” At block S8, the processor may determine changes in the color values of the returned light that has been detected.

The process to operate the blood pressure monitor may continue from block S8 to block S10, “Determine, by the processor, the blood pressure based on the changes in the color values.” At block S10, the processor may determine the blood pressure based on the changes in the color values. The processor may identify a first zone in the object based on the returned light. In some examples, processor 104 may perform calculations related to electrical signals which may correspond to the returned light detected by various photoreceptors 110 in order to identify the first zone. The first zone may be identified by a particular color value from among the color values. In some examples, processor 104 may perform a lookup to determine the particular color value. Processor 104 may determine that the particular color value may be maintained with respect to time. The processor may identify a second zone in the object based on the returned light. In various examples, processor 104 may perform calculations related to electrical signals which may correspond to the returned light detected by photoreceptors 110 in order to identify the second zone. In various examples, processor 104 may identify the second zone based on periodic variation of the color values C. In some examples, the variation of the color values C in the second zone may vary with a constant period with respect to time. The processor may determine the blood pressure based at least in part on the first and second zones. The processor may identify a boundary between the first and the second zone. In various examples, processor 104 may identify the boundary based on the position of particular photoreceptors which detect different characteristic functions. The processor may determine a resultant pressure at the boundary, where the resultant pressure may be calculated based on the applied pressure. The processor may determine a systolic blood pressure based on the boundary and the resultant pressure. The processor may identify a third zone in the object based on the returned light. In some examples, processor 104 may perform calculations related to electrical signals which may correspond to the returned light detected by various photoreceptors 110 in order to identify the third zone. The third zone may be identified by particular changes in the color values with respect to time. In various examples, processor 104 may determine that the particular changes in the color values may vary with a cycle of the heart rate of the subject. The processor may identify a boundary between the second zone and the third zone based on the position of various photoreceptors. The processor may determine a resultant pressure at the boundary. The resultant pressure may be calculated based on the applied pressure. The processor may determine a diastolic blood pressure based on the boundary and the resultant pressure. The processor may determine the blood pressure based at least in part on the area of the first and second zones. In some other examples, determining the blood pressure may be further based on an area of the first and/or second and/or third zones.

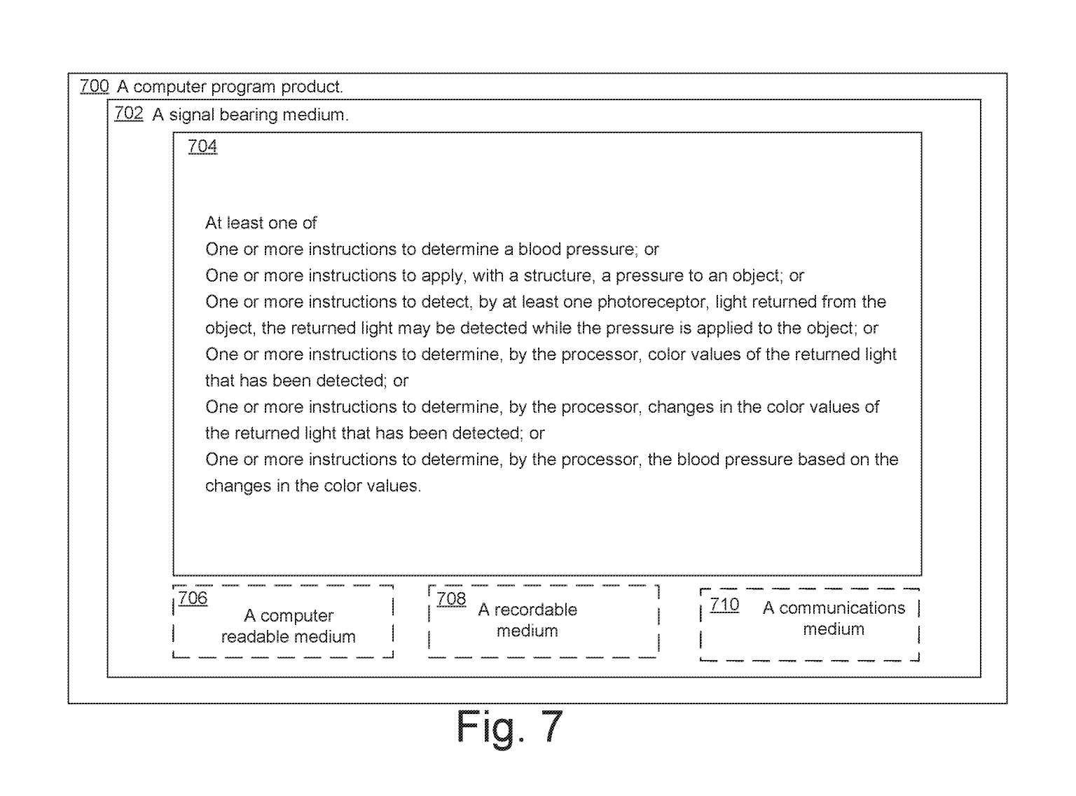

FIG. 7 illustrates an example computer program product 700 that can be utilized to operate a blood pressure monitor arranged in accordance with at least some embodiments described herein. Program product 700 may include a signal bearing medium 702. Signal bearing medium 702 may include one or more instructions 704 that, in response to execution by, for example, a processor, may provide the functionality and features described above with respect to FIGS. 1-6. Thus, for example, referring to device 100, processor 104 may perform or cause to be performed one or more of the blocks shown in FIG. 7 in response to instructions 704 conveyed to device 100 by medium 702.

In some implementations, signal bearing medium 702 may encompass a computer-readable medium 706, such as, but not limited to, a hard disk drive, a compact disc (CD), a digital video disk (DVD), a digital tape, memory, etc. In some implementations, signal bearing medium 702 may encompass a recordable medium 708, such as, but not limited to, memory, read/write (R/W) CDs, R/W DVDs, etc. In some implementations, signal bearing medium 702 may encompass a communications medium 710, such as, but not limited to, a digital and/or an analog communication medium (e.g., a fiber optic cable, a waveguide, a wired communications link, a wireless communication link, etc.). Thus, for example, program product 700 may be conveyed to one or more modules of the device 100 by an RF signal bearing medium 702, where the signal bearing medium 702 is conveyed by a wireless communications medium 710 (e.g., a wireless communications medium conforming with the IEEE 802.11 standard).

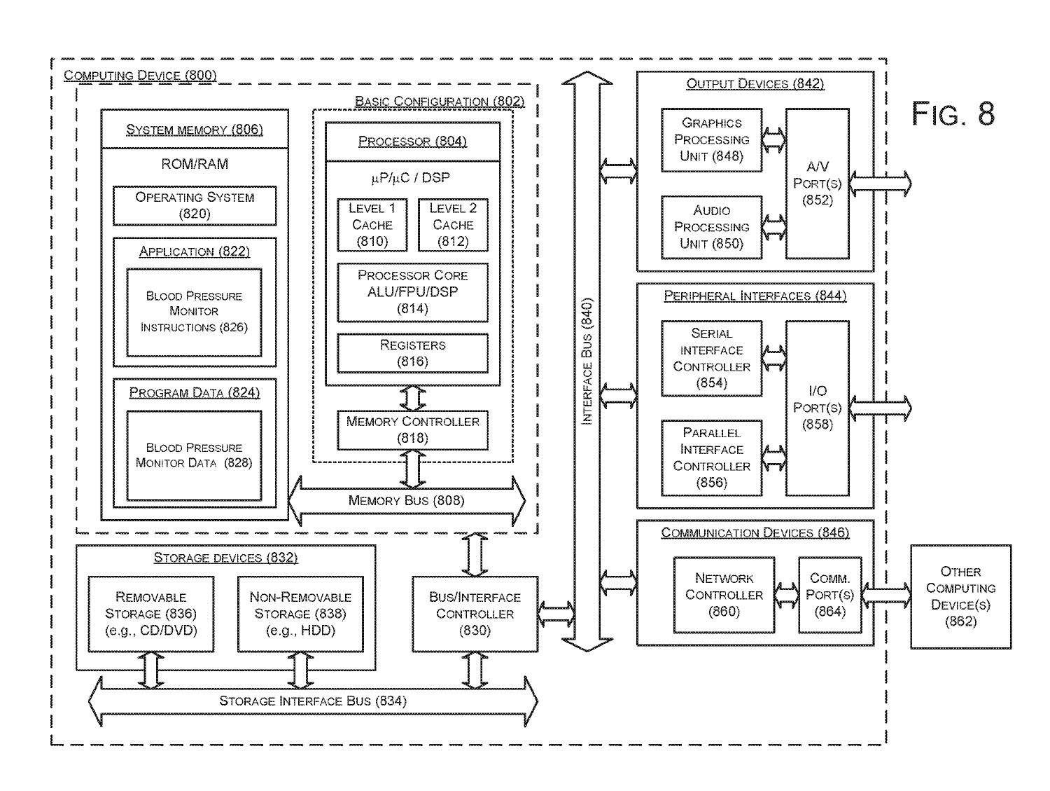

FIG. 8 is a block diagram illustrating an example computing device 800 that is arranged to implement or otherwise operate in conjunction with a blood pressure monitor, arranged in accordance with at least some embodiments described herein. For example, some elements of computing device 800 may be implemented within or as part of the devices described with respect to the embodiments shown in FIGS. 1-7. As another example, computing device 800 can be an external device, which receives signals/data from the devices described with respect to the embodiments shown in FIGS. 1-7, so as to determine blood pressure from the received data/signals. In a very basic configuration 802, computing device 800 typically includes one or more processors 804 (such as processor 104) and a system memory 806 (such as memory 106). A memory bus 808 may be used for communicating between processor 804 and system memory 806.

Depending on the desired configuration, processor 804 may be of any type including but not limited to a microprocessor (μP), a microcontroller (μC), a digital signal processor (DSP), or any combination thereof. Processor 804 may include one more levels of caching, such as a level one cache 810 and a level two cache 812, a processor core 814, and registers 816. An example processor core 814 may include an arithmetic logic unit (ALU), a floating point unit (FPU), a digital signal processing core (DSP core), or any combination thereof. An example memory controller 818 may also be used with processor 804, or in some implementations memory controller 818 may be an internal part of processor 804.

Depending on the desired configuration, system memory 806 may be of any type including but not limited to volatile memory (such as RAM), non-volatile memory (such as ROM, flash memory, etc.) or any combination thereof. System memory 806 may include an operating system 820, one or more applications 822, and program data 824. Application 822 may include blood pressure monitor instructions 826 that may be executable by a processor to perform or cause to be performed the functions and operations as described herein, including those described with respect to FIGS. 1-7 in connection with device 100. Program data 824 may include blood pressure monitor data 828 that may be useful to implement a blood pressure monitor as is described herein. For instance, program data 824 can include color values, values of optical/signal properties (such as intensity, wavelength, and frequency), boundary values, threshold or reference values, blood pressure reading values, C and time values, etc. In some embodiments, application 822 may be arranged to operate in cooperation with program data 824 and/or operating system 820 such that a blood pressure monitor may be provided. This described basic configuration 802 is illustrated in FIG. 8 by those components within the inner dashed line.

Computing device 800 may have additional features or functionality, and additional interfaces to facilitate communications between basic configuration 802 and any required devices and interfaces. For example, a bus/interface controller 830 may be used to facilitate communications between basic configuration 802 and one or more data storage devices 832 via a storage interface bus 834. Data storage devices 832 may be removable storage devices 836, non-removable storage devices 838, or a combination thereof. Examples of removable storage and non-removable storage devices include magnetic disk devices such as flexible disk drives and hard-disk drives (HDDs), optical disk drives such as compact disk (CD) drives or digital versatile disk (DVDs) drives, solid state drives (SSDs), and tape drives to name a few. Example computer storage media may include volatile and nonvolatile, removable and non-removable media implemented in any method or technology for storage of information, such as computer readable instructions, data structures, program modules, or other data.

System memory 806, removable storage devices 836 and non-removable storage devices 838 are examples of computer storage media. Computer storage media includes, but is not limited to, RAM, ROM, EEPROM, flash memory or other memory technology, CD-ROM, digital versatile disks (DVDs) or other optical storage, magnetic cassettes, magnetic tape, magnetic disk storage or other magnetic storage devices, or any other medium which may be used to store the desired information and which may be accessed by computing device 800. Any such computer storage media may be part of computing device 800.

Computing device 800 may also include an interface bus 840 for facilitating communication from various interface devices (e.g., output devices 842, peripheral interfaces 844, and communication devices 846) to basic configuration 802 via bus/interface controller 830. Example output devices 842 include a graphics processing unit 848 and an audio processing unit 850, which may be configured to communicate to various external devices such as a display or speakers via one or more A/V ports 852. Example peripheral interfaces 844 include a serial interface controller 854 or a parallel interface controller 856, which may be configured to communicate with external devices such as input devices (e.g., keyboard, mouse, pen, voice input device, touch input device, etc.) or other peripheral devices (e.g., printer, scanner, etc.) via one or more I/O ports 858. An example communication device 846 includes a network controller 860, which may be arranged to facilitate communications with one or more other computing devices 862 over a network communication link via one or more communication ports 864.

The network communication link may be one example of a communication media. Communication media may typically be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR) and other wireless media. The term computer readable media as used herein may include both storage media and communication media.

Computing device 800 may be implemented as a portion of a small-form factor portable (or mobile) electronic device such as a cell phone, a personal data assistant (PDA), a personal media player device, a wireless web-watch device, a personal headset device, an application specific device, or a hybrid device that include any of the above functions. Computing device 800 may also be implemented as a personal computer including both laptop computer and non-laptop computer configurations.

The present disclosure is not to be limited in terms of the particular embodiments described in this application, which are intended as illustrations of various aspects. Many modifications and variations can be made without departing from its spirit and scope. Functionally equivalent methods and apparatuses within the scope of the disclosure, in addition to those enumerated herein, will be apparent from the foregoing descriptions. Such modifications and variations are intended to fall within the scope of the appended claims. The present disclosure is to be limited only by the terms of the appended claims, along with the full scope of equivalents to which such claims are entitled. This disclosure is not limited to particular methods, reagents, compounds compositions or biological systems, which can, of course, vary. The terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

In general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). If a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “ a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g.,“a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

For any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, a range includes each individual member. Thus, for example, a group having 1-3 Cells refers to groups having 1, 2, or 3 Cells. Similarly, a group having 1-5 Cells refers to groups having 1, 2, 3, 4, or 5 Cells, and so forth.

While various aspects and embodiments have been disclosed herein, other aspects and embodiments are possible. The various aspects and embodiments disclosed herein are for purposes of illustration and are not intended to be limiting, with the true scope and spirit being indicated by the following claims.

1. A device, comprising:
a processor;
a structure effective to be in communication with the processor, wherein the structure is effective to apply pressure to an object; and
at least one photoreceptor effective to be in communication with the processor, wherein the at least one photoreceptor is located in the device so as to be effective to detect light returned from the object while the pressure is applied, and wherein the processor is effective to:
determine color values of the returned light,
determine changes in the color values of the returned light, and
determine a blood pressure based on the changes in the color values.

2. The device of claim 1, wherein the color values include at least first color values and second color values, and wherein to determine the blood pressure, the processor is effective to:
identify a first zone in the object based on the returned light, wherein the first zone is identified based on a first characteristic function, and wherein the first characteristic function relates the first color values to time;
identify a second zone in the object based on the returned light, wherein the second zone is identified based on a second characteristic function, wherein the second characteristic function relates the second color values to time, and wherein the first characteristic function is different from the second characteristic function; and
determine the blood pressure based on the first zone and the second zone.

3. The device of claim 1, wherein the at least one photoreceptor comprises a plurality of photoreceptors that are arranged annularly around the structure.

4. The device of claim 1, wherein the at least one photoreceptor comprises a plurality of photoreceptors that are arranged so as to extend linearly away from the structure.

5. The device of claim 1, further comprising a wristband, wherein the processor, the structure, and the at least one photoreceptor are located within the wristband.

6. The device of claim 1, wherein:
the device is included in augmented reality glasses, and
the structure is arranged in the augmented reality glasses so as to be in contact with the object.

7. The device of claim 1, wherein the object includes skin of a subject, wherein the device includes a surface effective to abut the skin of the subject, and wherein the structure is effective to extend from, and retract into, the surface of the device to apply the pressure.

8. The device of claim 1, wherein the structure is arranged in a conical shape, and wherein the structure is effective to apply the pressure to the object at a constant rate.

9. The device of claim 1, further comprising a light source configured to be in communication with the processor, wherein the light source is effective to emit light toward the object, and wherein the light emitted toward the object is generated as the returned light.

10. A method to determine a blood pressure, the method comprising:
applying, by a structure, a pressure to an object;
detecting, by at least one photoreceptor, light returned from the object, wherein the returned light is detected while the pressure is applied to the object;
determining, by a processor, color values of the returned light that has been detected;
determining, by the processor, changes in the color values of the returned light that has been detected; and
determining, by the processor, the blood pressure based on the changes in the color values.

11. The method of claim 10, wherein the color values include at least first color values and second color values, and wherein determining the blood pressure comprises:
identifying a first zone in the object based on the returned light, wherein the first zone is identified based on a first characteristic function, wherein the first characteristic function relates the first color values to time, and wherein the first color values are maintained with respect to time;
identifying a second zone in the object based on the returned light, wherein the second zone is identified based on a second characteristic function, wherein the second characteristic function relates the second color values to time, and wherein the second color values vary periodically with a constant period with respect to time; and
determining the blood pressure based at least, in part, on the first zone and the second zone.

12. The method of claim 11, wherein determining the blood pressure based on the first zone and the second zone comprises:
identifying a boundary between the first zone and the second zone;
determining a resultant pressure at the boundary, wherein the resultant pressure is calculated based on the applied pressure; and
determining a systolic blood pressure based on the boundary and the resultant pressure.

13. The method of claim 11, wherein the color values further include third color values, and wherein determining the blood pressure comprises:
identifying a third zone in the object based on the returned light, wherein the third zone is identified based on a third characteristic function, wherein the third characteristic function relates the third color values to time, and wherein the third color values vary with a cycle of a heart rate in the third zone;
identifying a boundary between the second zone and the third zone;
determining a resultant pressure at the boundary, wherein the resultant pressure is calculated based on the applied pressure; and
determining a diastolic blood pressure based on the boundary and the resultant pressure.

14. The method of claim 11, wherein determining the blood pressure is based on an area of the first zone and the second zone.

15. A method to determine blood pressure, the method comprising:
depressing skin of a subject, by a structure, at a first pressure;
detecting, by a photoreceptor, first returned light from the subject, wherein the first returned light is detected while the first pressure is applied to the skin of the subject;
determining, by a processor, a first color of the first returned light;
determining, by the processor, a first color change based on a first change in the first color of the first returned light;
depressing the skin of the subject, by the structure, at a second pressure, wherein the second pressure is different from the first pressure;
detecting, by the photoreceptor, second returned light from the subject, wherein the second returned light is detected while the second pressure is applied to the skin of the subject;
determining, by the processor, a second color of the second returned light;
determining, by the processor, a second color change based on a second change in the second color of the second returned light, wherein the second color change is different from the first color change; and
determining, by the processor, the blood pressure based on the first color change and the second color change.

16. The method of claim 15, wherein depressing the skin of the subject, by the structure, at the second pressure includes depressing the skin of the subject, by the structure, at a pressure greater than the first pressure.

17. The method of claim 15, further comprising emitting light toward the subject from a light source, wherein the light source is configured to be in communication with the processor.

18. The method of claim 15, wherein determining the blood pressure is based on an area of the structure that is pressed against the skin.

19. The method of claim 15, wherein detecting the first returned light includes detecting the first returned light by the photoreceptor, which is arranged inside the structure.

20. An apparatus to monitor blood pressure, the apparatus comprising:
a surface;
a structure configured to deploy from the surface and effective to apply pressure to skin of a subject in response to the surface being in proximity to the skin;
a light source coupled to the surface and effective to emit first light toward the skin of the subject; and
at least one photoreceptor coupled to the surface and located on the surface so as to be effective to:
detect second light returned from the skin of the subject while the pressure is applied; and
generate an output electrical signal that represents the detected second light,
wherein a color of the second light changes in response to the applied pressure, wherein the at least one photoreceptor is effective to change a characteristic of the output electrical signal in response to the changed color of the second light, and wherein the changed characteristic of the output electrical signal is usable to determine a blood pressure of the subject.

21-23. (canceled)

24. The apparatus of claim 20, wherein the structure comprises a needle with a dull point.

25. The apparatus of claim 20, further comprising a deployment component coupled to the structure and effective to cause the structure to extend to apply the pressure to the skin, and to cause the structure to retract to reduce the applied pressure to the skin.

26. The apparatus of claim 25, wherein the deployment component includes a motor.

27. The apparatus of claim 20, wherein the surface comprises a surface of a wearable device, and wherein the apparatus further comprises a processor included in the wearable device and effective to:
control an operation of the structure, the light source, and the at least one photoreceptor; and
determine the blood pressure based on the changed characteristic of the output electrical signal.

28. The apparatus of claim 20, wherein the surface comprises a surface of a wearable device, and wherein the apparatus further comprises a processor located externally to the wearable device and effective to communicate with the at least one photoreceptor to determine the blood pressure based on the changed characteristic of the output electrical signal.
